(19) United States
(12) Patent Application Publication
(10) Pub. No.: US 2014/0079744 A1
(43) Pub. Date: Mar. 20, 2014

(54) Even Deposition and Low Rub-Off Compositions

(71) Applicants: Pravin Shah, Trumbull, CT (US); Anjing Lou, Trumbull, NJ (US)

(72) Inventors: Pravin Shah, Trumbull, CT (US); Anjing Lou, Trumbull, NJ (US)

(73) Assignee: CONOPCO, INC., D/B/A UNILEVER, Englewood Cliffs, NJ (US)

(21) Appl. No.: 13/622,636

(22) Filed: Sep. 19, 2012

(51) Int. Cl. A61K 8/06; A61Q 19/00; A61K 8/19; A61Q 1/02; A61K 8/31; A61K 8/87

(52) U.S. Cl. 424/401; 424/63

(57) ABSTRACT

Improved deposition and low rub-off compositions are described. The compositions are emulsions with aqueous-based film-forming resins in the water phase and particle with hydrophobic material in the oil phase of the emulsion. The compositions are low rub-off after being topically applied.

### FIELD OF THE INVENTION

The present invention is directed to an even deposition and low rub-off compositions. More particularly, the invention is directed to a composition that comprises both particle comprising hydrophobic material and optionally active, as well as a film forming resin. The composition unexpectedly results in excellent active deposition and is not easy to rub-off. Moreover, the composition surprisingly does not result in a topical film that easily separates and peels from skin after being applied, notwithstanding the fact that aqueous-based film forming resin within the water phase of an emulsion is used with particle comprising hydrophobic material within the oil phase of the emulsion.

### BACKGROUND OF THE INVENTION

Skin appearance is a major indicia of looking healthy. Skin color, the lack of wrinkles and a complexion free of blemishes certainly makes one look and feel better.

Make-up formulas, for example, have been designed to mimic a person's skin tones. High loadings of colorants are optical ingredients necessary for such formulae. While formulae loaded with colorants achieve a covering purpose, high levels of colorants can negatively interfere with sensory attributes and active deposition. Moreover, formulae high in colorants can often, and undesirably, rub-off on clothing as well as other fabrics, including upholstery. Other formulae that provide good coverage of skin imperfections rely on the soft focus effect and utilize metal oxides, like titanium dioxide. These formulas whiten skin and hide blemishes but are easy to rub-off after application and usually result in an undesirable ashen appearance.

There is an increasing interest to develop compositions that are suitable to effectively deposit active on skin while at the same time being low rub-off and deplete of separation and peeling characteristics often associated with compositions classified as low rub-off. This invention, therefore, is directed to a low rub-off composition comprising both a particle comprising hydrophobic material and optionally active, as well as a film forming resin. The composition unexpectedly results in excellent active deposition and is low rub-off after application notwithstanding the fact that aqueous-based film forming resin within the water phase of an emulsion is used along with particle comprising hydrophobic material within the oil phase of the emulsion.

### ADDITIONAL INFORMATION

Efforts have been disclosed for making topical compositions. In U.S. Patent Application Publication No. 2009/0155373, cosmetic compositions and a method to impart a healthy appearance to skin are described.

In U.S. Patent Application Publication No. 2003/0021815, cosmetic compositions comprising at least one continuous liquid fatty phase containing at least one hydrocarbon-based oil are described whereby the same are structured with at least one polymer chosen from polyurethanes, polyurethaneureas and polyureas.

Still other efforts have been disclosed for making compositions suitable for topical application. In U.S. Patent Application Publication No. 2009/0155321, compositions with encapsulated coloring agents are described.

None of the additional information above describes a composition comprising a hydrophobic particle comprising hydrophobic material and optionally an active, as well as a film forming resin as claimed in this invention.

### SUMMARY OF THE INVENTION

In a first aspect, the present invention is directed to an improved deposition and low rub-off composition, the composition being an emulsion comprising:
(a) in an oil phase, a particle comprising hydrophobic material and optionally comprising an active; and
(b) in an aqueous phase, an aqueous based film forming resin

wherein combined weight of hydrophobic material in the particle and active in the particle/weight of film forming resin in the composition (m+a/r) is greater than or equal to 0.05.

In a second aspect, the present invention is directed to a method for improving a skin characteristic by applying topically the composition of the first aspect of this invention.

All other aspects of the present invention will more readily become apparent upon considering the detailed description and examples which follow.

Skin, as used herein, is meant to include skin on the face, neck, chest, back, arms (including underarms), hands, legs, buttocks and scalp. Particle comprising hydrophobic material, as used herein, is meant to mean a particle (or bead) that has a diameter from about 75 to about 525 microns where diameter is meant to mean the longest measurable (cross-sectional) distance in the event the particle is not a perfect sphere. Such a particle is, overall, hydrophobic. Active is meant to be embedded within the particle comprising hydrophobic material (like petrolatum) to form the preferred particle, and preferably, homogeneously mixed therein. Low rub-off, as used herein, generally means a composition capable of remaining on skin after normal contact with clothing worn. Active, as used herein, is meant to include a component that improves a skin characteristic and/or benefits skin wherein the same can be, and preferably, is an active in a leave-on composition, and most preferably, a cream, lotion, balm, deodorant, or gel. The weight ratio m+a/r means the amount by weight of hydrophobic material in the particle plus the amount by weight active in the particle/weight of film forming resin in the composition of this invention. Weight of film forming resin means the weight of resin used in the aqueous phase of the composition where the amount of resin (i.e., non-water soluble polymer) does not include the weight of the aqueous phase. Particle, as used herein, is meant to mean a single particle as well as an agglomerate of large particle made of smaller particles.

Comprising, as used herein, is meant to include consisting essentially of and consisting of. For the avoidance of doubt, therefore, the particle of this invention may consist essentially of or consist of hydrophobic material and optionally active. Emulsion means oil-in-water emulsion and may be interchanged with composition of this invention, where the composition of this invention is often one that is employed to evenly deposit active which is a colorant. All ranges identified herein are meant to include all ranges subsumed therein if, for example, reference to the same is not explicitly made.

### DETAILED DESCRIPTION OF THE PREFERRED EMBODIMENTS

The only limitation with respect to the particle comprising hydrophobic material and optional active used in this invention is that the same can be used in a topical composition and has a diameter from about 75 to about 525 microns, and preferably, from about 100 to about 450 microns, and most preferably, from about 150 to about 300 microns, including all ranges subsumed therein.

In an often preferred embodiment, the weight of hydrophobic material in the particle makes up from about 1.5 to about 9 times, and most preferably, from about 1.5 to about 7 times, and optimally, from about 2 to about 5 times more than the weight of active (i.e., when used) in the particle, based on total weight of the particle and including all ranges subsumed therein. The total weight of hydrophobic material in the composition is often from about 0.75 to about 10.0%, and preferably, from about 1.5 to about 6.0%, and most preferably, from about 1.5 to about 4.0% by weight, based on total weight of the composition of this invention and including all ranges subsumed herein. The total weight of optional active in the particle comprising hydrophobic material in the composition, when used, is often from about 0.02 to about 7.5%, and preferably, from about 0.05 to about 6.5%, and most preferably, from about 0.1 to about 5.0% by weight, based on total weight of the composition and including all ranges subsumed therein.

The hydrophobic material used for particle formation may comprise, for example, petrolatum, high molecular weight isoparaffins, polyalphaolefins, siliconized waxes like siliconized beeswax, shea butter, carnauba, silicones, silicone elastomer, glycerides (like sunflower seed oil, soybean oil, safflower oil), mixtures thereof or the like (consistent with the below melting points). The hydrophobic material or combination of hydrophobic material used for making particle of this invention is limited only to the extent that the materials may be topically applied, and preferably, has a melting point from about 40° to about 50° C., and preferably, from about 42° to about 48° C., including all ranges subsumed therein. In another often desired embodiment, the hydrophobic material of the particle comprises at least 50% by weight petrolatum, and preferably, from about 75 to about 100% by weight petrolatum as the hydrophobic material, and most preferably, 100% by weight petrolatum based on total weight of the hydrophobic material in the particle and including all ranges subsumed therein. It is within the scope of this invention for the particle comprising hydrophobic material to consist of petrolatum as the hydrophobic material. Moreover, the particle comprising hydrophobic material preferably comprises from about 50 to about 100%, and most preferably, from about 75% to 100% by weight hydrophobic material with or without active.

When making particle of the present invention, hydrophobic material is heated from about 40° to about 65° C., and preferably, from about 42° to about 60° C., and most preferably, from about 48° to 55° C., including all ranges subsumed therein. In an optional but preferred embodiment, active is combined with hydrophobic material and mixed. Mixing and heating is controlled and should continue until a preferably homogeneous composition of active and hydrophobic material is produced.

The resulting heated composition comprising hydrophobic material (and optionally but preferably active) is now suitable for addition to an oil phase on its own or already in the emulsion of this invention (i.e., composition of the invention) wherein the emulsion is one which is an oil-in-water emulsion. When adding the heated composition with or without active to the oil phase (i.e., the oil phase being used for emulsion formation or already in the emulsion), the same should be added to the oil phase and mixed with moderate shear via conventional mixing techniques (e.g., mechanical paddle). The oil phase should typically be maintained at a temperature from about 25° to about 48° C., and preferably, from about 30° to about 45° C., and most preferably, from about 30° to about 40° C., including all ranges subsumed therein. Heated composition comprising hydrophobic material (and optionally active) should be maintained at a temperature that is higher than the temperature of the oil phase prior to combining. Stirring should be maintained at a rate and for a time that results in particle comprising hydrophobic material (and optionally active) having a diameter described herein within the oil phase.

In an especially preferred embodiment, the m+a/r ratio is greater than 0.05 and less than 35, and most preferably, from about 0.1 to about 6, including all ranges subsumed therein. Optimally, the m+a/r ratio is from about 0.2 to about 1.0, and most optimally, the m+a/r ratio is from about 0.25 to about 0.5, including all ranges subsumed therein.

The only limitation with respect to the type of active that may optionally be used in this invention is that the active is one that can be mixed with particle comprising hydrophobic material (like, for example, petrolatum), and preferably, homogenously mixed within the hydrophobic particle. Such actives may be water or oil soluble or the actives used may be a mixture of water and oil soluble actives. Typically the active, when used, may be a particle itself having a diameter from about 100 to about 1200 nm, and preferably, from about 280 to about 1000 nm, and most preferably, from about 300 to about 650 nm, including all ranges subsumed therein. Consistent with the diameter for the particle comprising hydrophobic material, diameter for the active (if applicable) is meant to mean the longest measurable distance of a cross-section of the solid active in the event the same is not a perfect sphere.

In an optional embodiment, water soluble active is present within the particle comprising hydrophobic material and in the oil phase of the emulsion when slow release of the active is desired and/or when isolation from other actives in the water phase of the emulsion is desired. In a preferred embodiment, water soluble active is present in the water phase, and oil soluble active is present in the oil phase of the emulsion (and preferably carried in with the particle described in this invention). It is within the scope of this invention for active to be added to the water phase and/or oil phase of the emulsion without being carried by particle. Such active, not carried with particle, can be used with or in lieu of active carried with particle.

Actives suitable for use in this invention are meant to include but not be limited to opacifiers, colorants, skin lightening agents, moisturizers, sunscreens, plant extracts, anti-inflammatories, surfactants, wrinkle reducing agents, mixtures thereof or the like.

The aqueous-based film forming resin suitable for use in this invention can comprise acrylate, polymers of ether cellulose polyurethane, polyethylene glycols, polyvinyl pyrrolidone, polyvinyl alcohol, silicone polymer as well as mixtures, copolymers, ter-polymers and blends thereof. Preferably, non-water soluble resin (i.e., film forming resin) is dispersed in a water base before use so that the same may be homogeneously used within the water phase of the emulsion. Such dispersions are commercially available and may be made with or without emulsifiers. Often, polyoxyethylene side chain units present on the polymer or combination of polymers employed results in the desired aqueous-based water soluble resin. In a preferred embodiment, the most desired aqueous-based resin is an aqueous-based polyurethane resin sold under the name Baycusan® by Bayer Material Science. Typically, such aqueous-based film forming resins are from about 30 to 60%, and preferably, from 35 to 55%, and most preferably, from 40 to 50% by weight polymer or resin, based on total weight of the aqueous-based dispersion and including all ranges subsumed therein. The weight of resin (excluding the aqueous phase) used in this invention typically makes up from about 0.5 to 12%, and preferably, from about 1 to 10%, and most preferably from about 2 to 9% by weight of the composition of the invention, based on total weight of the composition and including all ranges subsumed therein. Typically, therefore, from about 1.5 to about 20% by weight aqueous-based resin in dispersion or aqueous phase is added to the composition of this invention, including all ranges subsumed therein. Traditional methods for making aqueous-based film forming resins are described, for example, in U.S. Pat. Nos. 3,310,416, 3,388,087 and 3,905,929, the disclosures of which are incorporated herein by reference.

Compositions (or oil-in-water emulsions) of the present invention may typically include cosmetically acceptable carrier components in addition to the water, oil, particles and resins described herein. Water, nevertheless, is the most preferred carrier. Amounts of water may range from about 1 to about 98%, and preferably, from about 5 to about 90%, and most preferably, from about 35 to about 80%, and optimally, from about 40 to about 75% by weight, based on total weight of the composition and including all ranges subsumed therein.

Cosmetically acceptable carriers suitable for use in this invention may include mineral oils, silicone oils, synthetic or natural esters, and alcohols. Amounts of these materials may range from about 0.1 to about 50%, and preferably, from about 0.1 to about 30%, and most preferably, from about 1 to about 20% by weight of the composition, including all ranges subsumed therein.

Silicone oils may be divided into the volatile and non-volatile variety. The term “volatile” as used herein refers to those materials which have a measurable vapor pressure at ambient temperature. Volatile silicone oils are preferably chosen from cyclic or linear polydimethylsiloxanes containing from about 3 to about 9, and preferably, from about 4 to about 5 silicon atoms.

Linear volatile silicone materials generally have viscosities of less than about 5 centistokes at 25° C. while cyclic materials typically have viscosities of less than about 10 centistokes.

Nonvolatile silicone oils useful as carrier material include polyalkyl siloxanes, polyalkylaryl siloxanes and polyether siloxane copolymers. The essentially non-volatile polyalkyl siloxanes useful herein include, for example, polydimethylsiloxanes (like dimethicone) with viscosities of from about 5 to about 100,000 centistokes at 25° C. Silicone oils (especially, Dimethicone 35 to 75 centistokes) suitable for use are often made commercially available from Dow Corning are preferred.

Among suitable esters are:
(1) Alkenyl or alkyl esters of fatty acids having 10 to 20 carbon atoms like isopropyl palmitate, isopropyl isostearate, isononyl isonanonoate, oleyl myristate, isopropyl myristate, oleyl stearate, and oleyl oleate;
(2) Ether-esters such as fatty acid esters of ethoxylated fatty alcohols;
(3) Polyhydric alcohol esters such as ethylene glycol mono- and di-fatty add esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters;
(4) Wax esters such as beeswax, spermaceti, myristyl myristate, stearyl stearate; and
(5) Sterol esters, of which soya sterol and cholesterol fatty acid esters are examples thereof.

Emulsifiers may be present in the composition of the present invention. Total concentration of the emulsifier may range from about 0.1 to about 40%, and preferably, from about 1 to about 20%, and most preferably, from about 1 to about 5% by weight of the composition, including all ranges subsumed therein. The emulsifier may be selected from the group consisting of anionic, nonionic, cationic and amphoteric actives. Particularly preferred nonionic actives are those with a C₁₀-C₂₀ fatty alcohol or acid hydrophobe condensed with from about 2 to about 100 moles of ethylene oxide or propylene oxide per mole of hydrophobe; C₂-C₁₀ alkyl phenols condensed with from 2 to 20 moles of alkylene oxide; mono- and di-fatty acid esters of ethylene glycol; fatty acid monoglyceride; sorbitan, mono- and di-C₈-C₂₀ fatty acids; and polyoxyethylene sorbitan as well as combinations thereof. Alkyl polyglycosides and saccharide fatty amides (e.g. methyl gluconamides) are also suitable nonionic emulsifiers.

Preferred anionic emulsifiers include alkyl ether sulfate and sulfonates, alkyl sulfates and sulfonates, alkylbenzene sulfonates, alkyl and dialkyl sulfosuccinates, C₈-C₂₀ acyl isethionates, C₈-C₂₀ alkyl ether phosphates, alkylethercarboxylates and combinations thereof.

Cationic emulsifiers that may be used include, for example, palmitamidopropyltrimonium chloride, distearyldimonium chloride and mixtures thereof. Useful amphoteric emulsifiers include cocoamidopropyl betaine, C₁₂-C₂₀ trialkyl betaines, sodium lauroamphoacetate, and sodium laurodiamphoacetate or a mixture thereof.

Other generally preferred emulsifiers include glyceryl stearate, glycol stearate, stearamide AMP, PEG-100 stearate, cetyl alcohol as well as emulsifying/thickening additives like hydroxyethylacrylate/sodium acryloyldimethyl taurates copolymer/squalane and mixtures thereof.

Emulsion stabilizers generally classified as vegetable based liquids may also be used. Preferred stabilizers are sold under the name Oilwax LC and made available commercially by Lotioncrafter.

Preservatives can desirably be incorporated into the compositions of this invention to protect against the growth of potentially harmful microorganisms. Suitable traditional preservatives for compositions of this invention are alkyl esters of para-hydroxybenzoic acid. Other preservatives which have more recently come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preferred preservatives are iodopropynyl butyl carbamate, phenoxyethanol, methyl paraben, propyl paraben, imidazolidinyl urea, sodium dehydroacetate and benzyl alcohol. The preservatives should be selected having regard for the use of the composition and possible incompatibilities between the preservatives and other ingredients in the emulsion. Preservatives are preferably employed in amounts ranging from about 0.01% to about 2% by weight of the composition, including all ranges subsumed therein.

Thickening agents may optionally be included in compositions of the present invention. Particularly useful are the polysaccharides. Examples include starches, natural/synthetic gums and cellulosics. Representative of the starches are chemically modified starches such as sodium hydroxypropyl starch phosphate and aluminum starch octenylsuccinate. Tapioca starch is often preferred. Suitable gums include xanthan, sclerotium, pectin, karaya, arabic, agar, guar, carrageenan, alginate and combinations thereof. Suitable cellulosics include hydroxypropyl cellulose, hydroxypropyl methylcellulose, ethylcellulose and sodium carboxy methylcellulose. Synthetic polymers are yet another class of effective thickening agent. This category includes crosslinked polyacrylates such as the Carbomers, polyacrylamides such as Sepigel® 305 and taurate copolymers such as Simulgel EG® and Aristoflex® AVC, the copolymers being identified by respective INCI nomenclature as Sodium Acrylate/Sodium Acryloyldimethyl Taurate and Acryloyl Dimethyltaurate/Vinyl Pyrrolidone Copolymer. Another preferred synthetic polymer suitable for thickening is an acrylate-based polymer made commercially available by Seppic and sold under the name Simulgel INS100.

Amounts of the thickener, when used, may range from about 0.001 to about 5%, and preferably, from about 0.1 to about 3%, and most preferably, from about 0.2 to about 1.5% by weight of the composition including all ranges subsumed therein.

Conventional humectants may be employed in the present invention. These are generally polyhydric alcohol-type materials. Typical polyhydric alcohols include glycerol (i.e., glycerine or glycerin), propylene glycol, dipropylene glycol, polypropylene glycol, polyethylene glycol, sorbitol, hydroxypropyl sorbitol, hexylene glycol, 1,3-butylene glycol, isoprene glycol, 1,2,6-hexanetriol, ethoxylated glycerol, propoxylated glycerol and mixtures thereof. Most preferred is glycerin, propylene glycol or a mixture thereof. The amount of humectant employed may range anywhere from 0.5 to 20%, preferably between 1 and 15% by weight of the composition.

Fragrances, colorants, fixatives and abrasives may optionally be included in compositions of the present invention. Each of these substances may range from about 0.05 to about 5%, preferably between 0.1 and 3% by weight.

Turning to the actives suitable for use herein, the same can include opacifiers like TiO₂ and ZnO and colorants like iron oxide red, yellow and black. Such opacifiers and colorants typically have a particle size from 50 to 1200 nm, and preferably, from 50 to 350 nm.

To enhance skin moisturization, actives classified as cationic ammonium compounds may optionally be used in the compositions of this invention. Such compounds include salts of hydroxypropyltri(C₁-C₃ alkyl)ammonium mono-substituted-saccharide, salts of hydroxypropyltri(C₁-C₃ alkyl)ammonium mono-substituted polyols, dihydroxypropyltri(C₁-C₃ alkyl)ammonium salts, dihydroxypropyldi(C₁-C₃ alkyl)mono(hydroxyethyl)ammonium salts, guar hydroxypropyl trimonium salts, 2,3-dihydroxypropyl tri(C₁-C₃ alkyl or hydroxalkyl)ammonium salts or mixtures thereof. In a most preferred embodiment and when desired, the cationic ammonium compound employed in this invention is the quaternary ammonium compound 1,2-dihydroxypropyltrimonium chloride. If used, such compounds typically make up from about 0.01 to about 30%, and preferably, from about 0.1 to about 15% by weight of the composition.

When cationic ammonium compounds are used, preferred additional active for use with the same are moisturizing agents such as substituted ureas like hydroxymethyl urea, hydroxyethyl urea, hydroxypropyl urea; bis(hydroxymethyl)urea; bis(hydroxyethyl)urea; bis(hydroxypropyl)urea; N,N′-dihydroxymethyl urea; N,N′-di-hydroxyethyl urea; N,N′-di-hydroxypropyl urea; N,N,N′-tri-hydroxyethyl urea; tetra(hydroxymethyl)urea; tetra(hydroxyethyl)urea; tetra(hydroxypropyl)urea; N-methyl-N′-hydroxyethyl urea; N-ethyl-N,N—N′-hydroxyethyl urea; N-hydroxypropyl-N′-hydroxyethyl urea and N,N′-dimethyl-N-hydroxyethyl urea or mixtures thereof. Where the term hydroxypropyl appears, the meaning is generic for either 3-hydroxy-n-propyl, 2-hydroxy-n-propyl, 3-hydroxy-1-propyl or 2-hydroxy-1-propyl radicals. Most preferred is hydroxyethyl urea. The latter is available as a 50% aqueous liquid from the National Starch & Chemical Division of ICI under the trademark Hydrovance.

Amounts of substituted urea, when used, in the composition of this invention range from about 0.01 to about 20%, and preferably, from about 0.5 to about 15%, and most preferably, from about 2 to about 10% based on total weight of the composition and including all ranges subsumed therein.

When cationic ammonium compound and substituted urea are used, in a most especially preferred embodiment at least from about 1 to about 15% glycerin external to the particle is used, based on total weight of the composition and including all ranges subsumed therein.

Compositions of the present invention may include vitamins as the desired active. Illustrative vitamins are Vitamin A (retinol) as well as retinol esters like retinol palmitate and retinol propionate, Vitamin B₂, Vitamin B₃ (niacinamide), Vitamin B₆, Vitamin C, Vitamin E, Folic Acid and Biotin. Derivatives of the vitamins may also be employed. For instance, Vitamin C derivatives include ascorbyl tetraisopalmitate, magnesium ascorbyl phosphate and ascorbyl glycoside. Derivatives of Vitamin E include tocopheryl acetate, tocopheryl palmitate and tocopheryl linoleate. DL-panthenol and derivatives may also be employed. Total amount of vitamins when present in compositions according to the present invention may range from 0.001 to 10%, preferably from 0.01% to 1%, optimally from 0.1 to 0.5% by weight of the composition.

Octadecenedioic acid, azelaic acid, ubiquinone, dihydroxyacetone (DHA) and mixtures thereof may also be used as actives in the composition of this invention. Such compounds, when used, typically make up from about 0.2 to 4.5%, and preferably, from about 0.5 to 3% by weight of the composition, including all ranges subsumed therein.

Other optional actives suitable for use in this invention include resveratrol, resorcinols like 4-ethyl resorcinol, 4-hexyl resorcinol, 4-phenylethyl resorcinol, dimethoxytoluoyl propyl resorcinol, 4-cyclopentyl resorcinol, 4-cyclohexylresorcinol, alpha- an/or beta-hydroxyacids, petroselinic acid, conjugated linoleic acid, octadecanoic acid, phenylethyl resorcinol (Symwhite 377 from Symrise), undecylenol phenylalanine (Seppi White from Seppic) mixtures thereof or the like. Such actives, when used, collectively make up from about 0.001 to about 12% by weight of the composition.

Desquamation promoters may be present. Illustrative are the alpha-hydroxycarboxylic acids, beta-hydroxycarboxylic acids. The term “acid” is meant to include not only the free acid but also salts and C₁-C₃₀ alkyl or aryl esters thereof and lactones generated from removal of water to form cyclic or linear lactone structures. Representative acids are glycolic and its derivatives, lactic and malic acids. Salicylic acid is representative of the beta-hydroxycarboxylic acids. Amounts of these materials when present may range from about 0.01 to about 15% by weight of the composition.

A variety of herbal extracts may optionally be included as actives in compositions of this invention. The extracts may either be water soluble or water-insoluble carried in a solvent which respectively is hydrophilic or hydrophobic. Water and ethanol are the preferred extract solvents. Illustrative extracts include those from green tea, yarrow, chamomile, licorice, aloe vera, grape seed, citrus unshui, willow bark, sage, thyme and rosemary. Soy extracts may be used and especially when it is desirable to include retinol.

Also optionally suitable for use include materials like chelators (e.g., EDTA), C₈₋₂₂ fatty acid substituted saccharides, lipoic acid, retinoxytrimethylsilane (available from Clariant Corp. under the Silcare 1M-75 trademark), dehydroepiandrosterone (DHEA) and combinations thereof. Ceramides (including Ceramide 1, Ceramide 3, Ceramide 3B and Ceramide 6) as well as pseudoceramides may also be useful. Occlusives like Oilwax LC are often desired. Amounts of these materials may range from about 0.000001 to about 10%, preferably from about 0.0001 to about 1% by weight of the composition.

Sunscreen actives may also be included in compositions of the present invention and carried by the particle comprising hydrophobic material as described herein. Particularly preferred are such materials as phenylbenzimidazole sulfonic acid (Ensulizole), ethylhexyl p-methoxycinnamate, available as Parsol MCX®, Avobenzene, available as Parsol 1789® and benzophenone-3, also known as Oxybenzone. Inorganic sunscreen actives may be employed such as microfine titanium dioxide, zinc oxide, polyethylene and various other polymers. Also suitable for use is octocrylene. Amounts of the sunscreen agents when present may generally range from 0.1 to 30%, preferably from 0.5 to 20%, optimally from 0.75 to 10% by weight.

Conventional buffers/pH modifiers may be used. These include commonly employed additives like sodium hydroxide, potassium hydroxide, hydrochloric acid, citric acid and citrate/citric acid buffers. In an especially preferred embodiment, the pH of the composition of this invention is from about 4 to about 8, and preferably, from about 4.25 to about 7.75, and most preferably, from about 6 to about 7.5, including all ranges subsumed therein. The composition of this invention may be a solid stick or bar. Viscosity of the composition of this invention is, however, preferably from about 1,000 to about 120,000 cps, and most preferably, from about 5,000 to 80,000 cps, taken at ambient temperature NS and a shear rate of 1 s⁻¹ with a strain controlled parallel plate rheometer made commercially available from suppliers like T.A. Instruments under the Ares name.

In an especially preferred embodiment, actives like dihydroxyacetone, colorants such as metal oxides, vitamins (e.g., niacinamide) and/or substituted ureas are homogeneously mixed within the particle comprising hydrophobic material. In a most especially preferred embodiment, the active homogeneously mixed within the particle comprising hydrophobic material is metal oxide like an iron oxide.

A wide variety of packaging can be employed to store and deliver the composition of this invention. Packaging is often dependent upon the type of personal care end-use. For instance, leave-on skin lotions and creams, shampoos, conditioners and shower gels generally employ plastic containers with an opening at a dispensing end covered by a closure. Typical closures are screw-caps, non-aerosol pumps and flip-top hinged lids. Packaging for antiperspirants, deodorants and depilatories may involve a container with a roll-on ball on a dispensing end. Alternatively these types of personal care products may be delivered in a stick composition formulation in a container with propel-repel mechanism where the stick moves on a platform towards a dispensing orifice. Metallic cans pressurized by a propellant and having a spray nozzle serve as packaging for antiperspirants, shave creams and other personal care products. Toilette bars may have packaging constituted by a cellulosic or plastic wrapper or within a cardboard box or even encompassed by a shrink wrap plastic film.

The following examples are provided to facilitate an understanding of the present invention. The examples are not intended to limit the scope of the claims.

### Example 1

| | | | Sample | Sample | Sample |
|---|---|---|---|---|---|
| | Ingredients | Control | 1 | 2 | 3 |
| Emul- | Deionized water | Balance | Balance | Balance | Balance |
| sion | Thickner | 1.00 | 1.00 | 1.00 | 1.00 |
| Phase | (Simugel) | | | | |
| | Opacifier | 0.50 | 0.50 | 0.50 | 0.50 |
| | Propylene glycol | 12.00 | 12.00 | 12.00 | 12.00 |
| | Preservative | 0.70 | 0.70 | 0.70 | 0.70 |
| | Disodium EDTA | 0.05 | 0.05 | 0.05 | 0.05 |
| | Citric acid | 0.10 | 0.10 | 0.10 | 0.10 |
| | Aqueous-based | 0.00 | 8.00 | 3.00 | 5.00 |
| | polyurethane | | | | |
| | resin | | | | |
| | Glycerylstearate | 2.40 | 2.40 | 2.40 | 2.40 |
| | Cetyl alcohol | 2.40 | 2.40 | 2.40 | 2.40 |
| | PEG-100 stearate | 1.20 | 1.20 | 1.20 | 1.20 |
| | Dimethicone | 3.00 | 3.00 | 3.00 | 3.00 |
| | 50 cst | | | | |
| | Isopropyl | 2.00 | 2.00 | 2.00 | 2.00 |
| | palmitate | | | | |
| | Oilwax LC | 1.00 | 1.00 | 1.00 | 1.00 |
| | occlusive | | | | |
| | Tapioca starch | 0.50 | 0.50 | 0.50 | 0.50 |
| | DHA | 2.50 | 2.50 | 2.50 | 2.50 |
| | Base color | 0.35 | 0.35 | 0.35 | 0.35 |
| Particle/ | Petroleum | 2.50 | 2.50 | 2.50 | 2.50 |
| active | Iron oxide red | 0.25 | 0.25 | 0.25 | 0.25 |
| phase | Iron oxide yellow | 0.10 | 0.10 | 0.10 | 0.10 |
| | Iron oxide black | 0.25 | 0.25 | 0.25 | 0.25 |

*Baycusan-C1004 made available by Bayer Material Science.

In this example, water and water soluble and dispersible ingredients were thoroughly mixed as were oil and oil soluble and dispersible ingredients to produce water phases and oil phases. Heat was used but no phases were heated above about 70° C. and dihydroxyacetone and fragrance were not added until cooling to about 35° C. was achieved. The oil phases for each sample were added to the water phases for each sample and thoroughly mixed in the presence of emulsifier. Produced were oil-in-water emulsions. Particles with optional active were made by combining petrolatum and active (i.e., iron oxide red, yellow and black). Moderate stirring was used and the particle/active phases were heated to about 50° C. Homogeneous mixtures of petrolatum and iron oxide were obtained.

The homogeneous mixtures of petrolatum and iron oxide were maintained at a temperature of about 48° C. and the same were added to emulsion at a temperature of about 33° C. Conventional stirring was employed to combine emulsion and homogeneous mixture wherein the resulting emulsions having aqueous-based resin in the aqueous phase and particle comprising hydrophobic material and active in the oil phase were produced with particle having a diameter of about 200 microns. The control composition was deplete of aqueous-based film forming resin.

### Example 2

The samples of Example 1 were assessed for rub-off and visually observed for evenness of application. The surprising results obtained may be found in the table below.

TABLE

| Sample | Delta E* | Evenness |
|---|---|---|
| Control | 5.2 | 4.5 |
| 1 | 0.9 | 2 |
| 2 | 1.5 | 2.5 |
| 3 | 1.3 | 2.5 |

Delta E*, color difference, was obtained after samples were applied to artificial skin substrates (23 cm×7 cm) with the finger and rubbed for 60 seconds. A film applicator (1u) was run through the surface to make a 25 um thick film. A piece of white fabric (r=3.5 cm) was applied to the bottom of a plastic disk (r=2.7 cm). Ten (10) minutes after application, the disk was placed on the surface of the substrate with a controlled loading pressure (30 g/cm2) and moved across the substrate surface with an apparatus (IKA Labortechnik) that dragged the disk at a speed of 2.5 cm/s. The substrate selected was solid grip liner, 2 mm thick, soft, visco-elastic and hydrophobic. Temperature was maintained at about 21° C. and relative humidity was about 25-40%. Color was used for an easy assessment of low rub-off and evenness of application. Delta E* was assessed by using a commercially available Hunter Lab color measurement instrument. The lower the Delta E* value, the less color change, and therefore, the less rub-off of the composition. Evenness of application was assessed by applying composition to skin wherein skilled panelists made visual assessment with results at 5 being poor and at 1 being outstanding. The results unexpectedly demonstrate that compositions made consistent with this invention unexpectedly resulted in even deposition and low rub-off characteristics, notwithstanding the fact that aqueous-based film forming resin was used in a water phase of an emulsion and particle comprising hydrophobic material was used in the oil phase of the emulsion. Moreover, subsequent to visual inspection the compositions made consistent with this invention were surprisingly easily washed off but did not peel off during normal activity.

1. A composition, the composition being an emulsion comprising:
(a) in an oil phase, a particle comprising hydrophobic material and a water soluble active;
(b) in an aqueous phase, an aqueous based film forming resin, and
(c) wherein the water soluble active is selected from the group consisting of water soluble, dihydroxyacetone, vitamin(s), substituted urea(s), cationic ammonium compounds, panthenol, resorcinol(s), alpha and beta hydroxyl acids and blends thereof; and
wherein weight of hydrophobic material in the particle and active in the panicle/weight of film forming resin in the composition (m+a/r) is greater than or equal to 0.05.

2. The composition according to claim 1 wherein (m+a/r) is greater than 0.05 to less than 35.

3. The composition according to claim 1 wherein (m+a/r) is from about 0.1 to about 6.0.

4. The composition according to claim 1 wherein particle comprising hydrophobic material has a diameter from about 75 to 525 microns.

5. (canceled)

6. The composition according to claim 1 wherein the hydrophobic material is petrolatum.

7. The composition according to claim 1 wherein the resin is polyurethane.

8. The composition according to claim 1 wherein the aqueous-based film forming resin is from about 30 to about 60% by weight resin.

9. (canceled)

10. The composition according to claim 1 wherein the active further comprises a colorant.

11. The composition according to claim 10 wherein the colorant comprises iron oxide.

12. The composition according to claim 1 wherein m+a/r is from about 0.2 to about 1.0.

13. The composition according to claim 1 wherein m+a/r is from about 0.25 to about 0.5.

14. A method for improving a skin characteristic comprising the step of applying topically to skin the composition of claim 1.
